SEQUENCE LISTING (8 sequences)

SEQ ID NO: 1 - DNA, 73 bp, Artificial Sequence
  Synthetic
gctaaccatgttcatgccttcttctttttcctacagctcctgggcaacgtgctggttgttgtgctgtctcatc

SEQ ID NO: 2 - DNA, 73 bp, Artificial Sequence
  Synthetic
ttcatgccattcaattttctgcgcttcaaaaatatcgttcaggccgctgccgtgatgatgatgatgatgtccc

SEQ ID NO: 3 - DNA, 78 bp, Artificial Sequence
  Synthetic
atgtcgggcctgaacgatatttttgaagcgcagaaaattgaatggcatgaaggcagcatgttcgtgtttctggtgctg

SEQ ID NO: 4 - DNA, 78 bp, Artificial Sequence
  Synthetic
cttcatgatgtccccataatttttggcagagggaaaaagatctgctagctcgagtcgcgacttaagatcgatgcggcc

SEQ ID NO: 5 - DNA, 71 bp, Artificial Sequence
  Synthetic
ggcagcggcctgaacgatatttttgaagcgcagaaaattgaatggcatgaataatgaaattcgagctcgcg

SEQ ID NO: 6 - DNA, 73 bp, Artificial Sequence
  Synthetic
gatgagacagcacaacaaccagcacgttgcccaggagctgtaggaaaaagaagaaggcatgaacatggttagc

SEQ ID NO: 7 - DNA, 70 bp, Artificial Sequence
  Synthetic
cttaagtcgcgactcgagctagcagatctttttccctctgccaaaaattatggggacatcatgaagcccc

SEQ ID NO: 8 - DNA, 76 bp, Artificial Sequence
  Synthetic
gctgccttcatgccattcaattttctgcgcttcaaaaatatcgttcaggcccgacatggtggcctttgccaaaatg

(19) United States
(12) Patent Application Publication
(10) Pub. No.: US 2023/0349829 A1
(43) Pub. Date: Nov. 2, 2023

(54) METHODS OF DIAGNOSTICS

(71) Applicant: TECHNION RESEARCH & DEVELOPMENT FOUNDATION LIMITED, Haifa (IL)

(72) Inventors: Yoav SHECHTMAN, Haifa (IL); Onit ALALOUF, Regba (IL); Lucien WEISS, Los Angeles, CA (US)

(21) Appl. No.: 18/016,932

(22) PCT Filed: Aug. 3, 2021
(86) PCT No.: PCT/IL2021/050936

§ 371 Date: Jan. 19, 2023

Related U.S. Application Data
(60) Provisional application No. 63/060,216, filed on Aug. 3, 2020.

(51) Int. Cl. G01N 21/64

(52) U.S. Cl. CPC G01N 21/6458; G01N 2021/6441

(57) ABSTRACT

The present invention is directed to a method for determining the presence of a first molecule, e.g., a protein, in a sample, wherein the first molecule has specific binding affinity to a second molecule, e.g., a protein. Further, the present invention is directed to a method for determining the presence of a particle, e.g., a viral particle, in sample. Further provided are a system and a computer readable medium configured for determination of the presence of a molecule in a sample.

### CROSS-REFERENCE TO RELATED APPLICATIONS

This application claims the benefit of priority to U.S. Provisional Patent Application No. 63/060,216, titled “METHODS OF DIAGNOSTICS”, filed Aug. 3, 2020, the contents of which are incorporated herein by reference in their entirety.

### FIELD OF INVENTION

The present invention is in the field of diagnostics including multicolor localization microscopy.

### BACKGROUND

The ability to detect ultra-low levels of molecules has great implications for diagnostics, therapeutics, and research. For example, early and continuous detection of antibodies is crucial to determine the progression of a disease, e.g., a viral disease, such as COVID19, and estimate future immunity in a population scale, define exposure and identify human donors for the generation of convalescent serum as therapeutic.

Currently, ELISA (enzyme-linked immunosorbent assay) is the most accurate quantitative platform for immunoassays, and specifically for total antibody detection. According to this assay, an antigen (e.g., viral recombinant protein; either bound to a surface or not) binds to antibodies in a sample, e.g., a subject's serum sample, and the bound complex is subsequently reported by a second antibody, or an antigen linked to an enzyme. The lower limit of detection with immunoassay technology is the upper femtomolar (10⁻¹³ M) to the attomolar range (10⁻¹⁶ M). Accordingly, this field still faces a challenge of early diagnosis in cases wherein antibodies and/or protein biomarkers are present in very low amounts.

There is still a great need for a specific, sensitive, fast, and cost-effective methodology for detecting ultra-low levels of molecules in a sample of a subject.

### SUMMARY

According to a first aspect, there is provided a method for determining the presence of a first molecule in a sample, wherein the first molecule has specific binding affinity to a second molecule, the method comprising the steps of: (a) labeling molecules of a sample suspect of comprising the first molecule with a first labeling agent; (b) contacting the sample comprising the labeled molecules from step (a) with second molecule labeled with a second labeling agent; and (c) determining, under flow conditions, the temporal localization of the first labeling agent and of the second labeling agent, wherein colocalization of the first labeling agent and the second labeling agent in at least two time points is indicative of the presence of the first molecule having specific binding affinity to the second molecule in the sample, thereby determining the presence of the first molecule in the sample.

According to another aspect, there is provided a method for determining the presence of a particle in sample, the method comprising the steps of: (a) contacting a sample suspected of comprising a particle with a labeled compound having specific binding affinity to the particle; and (b) determining the intensity of a signal generated by the labeled compound, wherein a detection of a signal above a predetermined threshold provided by a background is indicative of the presence of the particle in the sample, thereby determining the presence of the particle in the sample.

In some embodiments, the determining comprises generating a three-dimensional image based on a modified light path to provide the depth or color of any one of the first molecule labeled with the first labeling agent and the second molecule labeled with the second labeling agent.

In some embodiments, the any one of the first molecule and the second molecule is selected from the group consisting of: a peptide, a nucleic acid, and a small molecule.

In some embodiments, the first molecule is a biomarker indicative of any one of: cancer, brain injury or disease, inflammation, and an infectious disease.

In some embodiments, the first molecule, the second molecule, or both, are proteins.

In some embodiments, the first molecule being a protein is an antibody or a cytokine.

In some embodiments, the second molecule being a protein is an antigen.

In some embodiments, the antigen comprises a viral antigen.

In some embodiments, the first molecule, the second molecule, or both, are polynucleotides.

In some embodiments, the first molecule being a polynucleotide comprises a host polynucleotide or a pathogen polynucleotide.

In some embodiments, the polynucleotide comprises DNA, RNA, or a hybrid thereof.

In some embodiments, the first label, the second label, or both, are fluorescent labels.

In some embodiments, the flow conditions comprise microfluidics, diffusion, or both.

In some embodiments, the specific binding affinity is binding with a dissociation constant (K_{D}) ranging from 0.1 to 50 nM.

In some embodiments, the method further comprises determining the number of counts of the detected signal derived from the sample and being above the predetermined threshold, compared to the background, wherein an increase of at least 5% in the number of counts of the detected signal derived from the sample and being above the predetermined threshold, compared to the background, is indicative of the presence of the particle in the sample.

In some embodiments, the particle comprises a virus or a viral protein.

In some embodiments, the protein is a receptor or comprises a ligand binding domain.

In some embodiments, the labeled compound comprises a ligand of the protein and a dye.

In some embodiments, the dye comprises a fluorescent dye.

In some embodiments, the sample is derived from a subject.

In some embodiments, the sample derived from a subject comprises a cell, a tissue, and organ, a bodily fluid, or a fraction thereof, or any combination thereof, of the subject.

In some embodiments, the subject is exposed or is suspected of being exposed to an infectious agent.

In some embodiments, the infectious agent is selected from the group consisting of: a virus, a bacterium, a fungus, a unicellular parasite, and a microparasite.

In some embodiments, the subject is afflicted with a disease or an injury.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include a scheme and flowchart. (1A) A scheme of a non-limiting outline of a simple, fast, and sensitive antibody and virion detection by microscopy. Top: accurate antibody-antigen interaction detection includes labeling followed by immediate 3D two-color colocalization. Bottom: virus visualization requires the addition of fluorescent molecules that bind the spike proteins on the virus and give enhanced signal over the sample background. (1B) A flowchart describing non-limiting steps of a method as disclosed here.

FIG. 2 includes a micrograph showing validation of plasmid restriction products, as analyzed using gel (1%) electrophoresis.

FIG. 3 includes a micrograph showing validation of Spike PCR product, as analyzed using gel (1%) electrophoresis.

FIG. 4 includes a micrograph showing validation of plasmid restriction products, as analyzed using gel (1%) electrophoresis.

FIG. 5 includes a micrograph demonstrating protein-antibody interactions by fluorescence co-localization microscopy of a spike protein and an anti-spike antibody. Single (probably as a trimer) labeled spike protein (top PSF) is attached to an anti-spike antibody (bottom PSF) and is floating in the drop. Laser sequence: 640+561; 561; 640. The channels are shifted in the field of view where the top tilted PSF corresponds to the orange channel and bottom PSF corresponds to red channel and is less tilted.

FIGS. 6A-6B include a block diagram and a flowchart. (6A) A block diagram of a computer system according to some embodiments of the invention. (6B) A flowchart of a computer-based method for determining a presence of a first molecule in a sample, to be executed by the computer system of (6A), according to some embodiments of the invention.

### DETAILED DESCRIPTION

According to some embodiments, there is provided a method for determining the presence of a first molecule in a sample, wherein the first molecule has specific binding affinity to a second molecule, comprising the steps of: (a) providing a sample comprising labeled molecules and suspect of comprising the first molecule; (b) contacting the sample comprising labeled molecules from step (a) with the second molecule, wherein the second molecule is labeled with a second labeling agent; and (c) determining, under flow conditions, the temporal localization of the first labeling agent and of the second labeling agent.

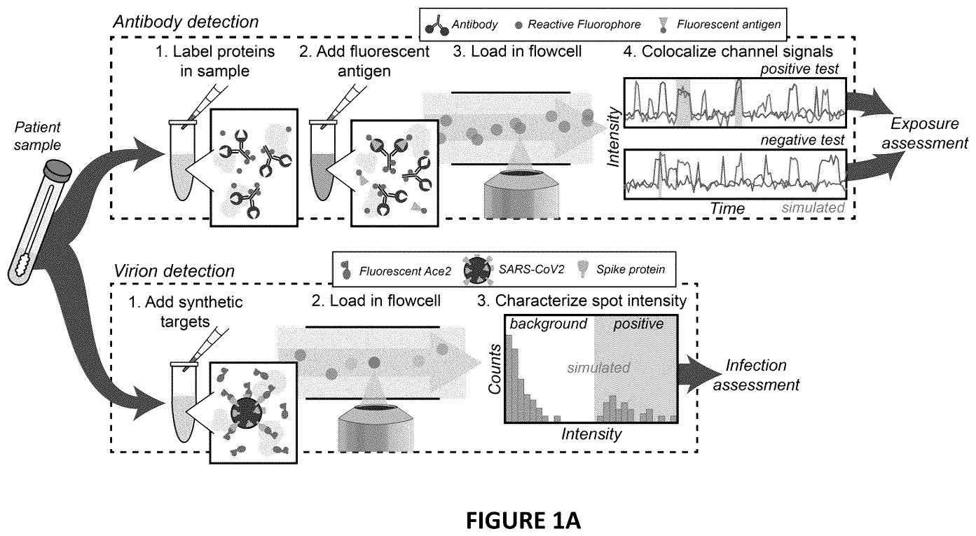

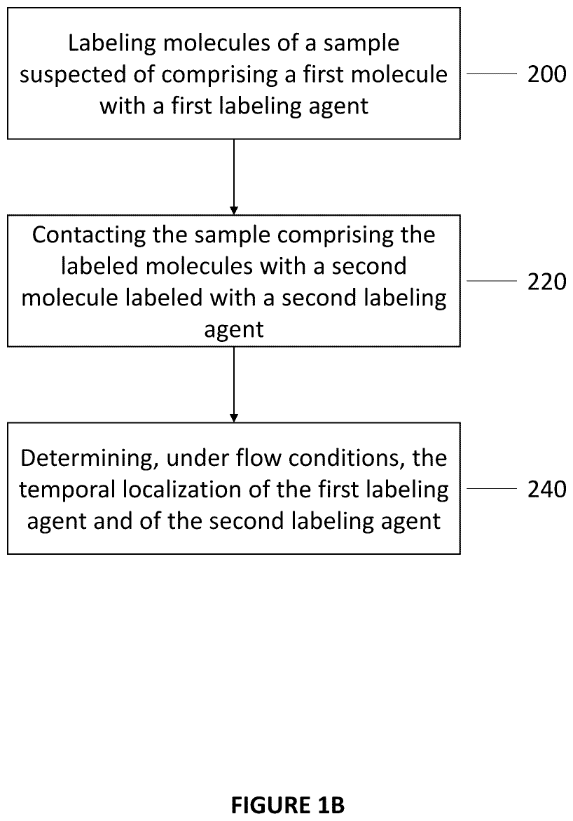

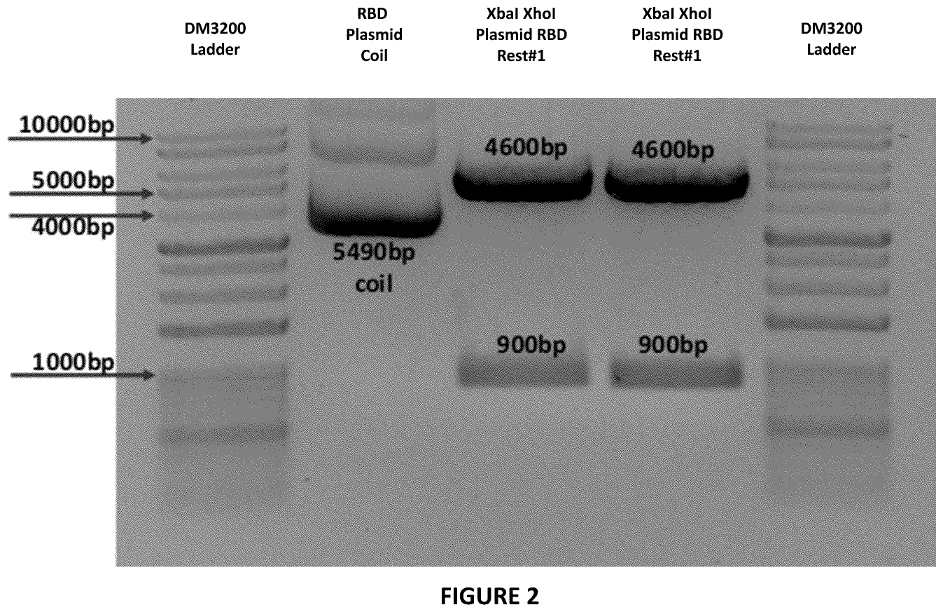

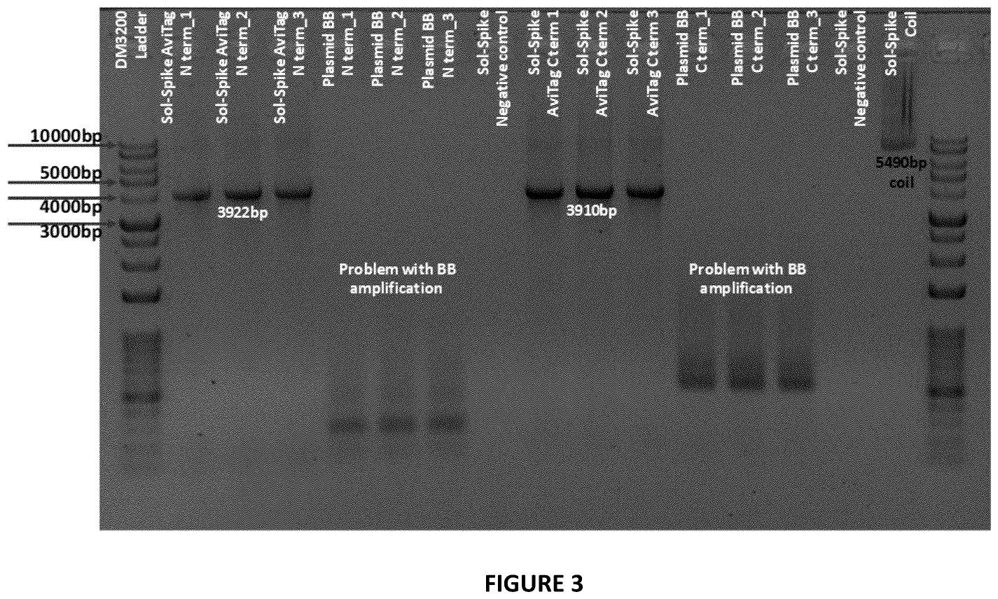

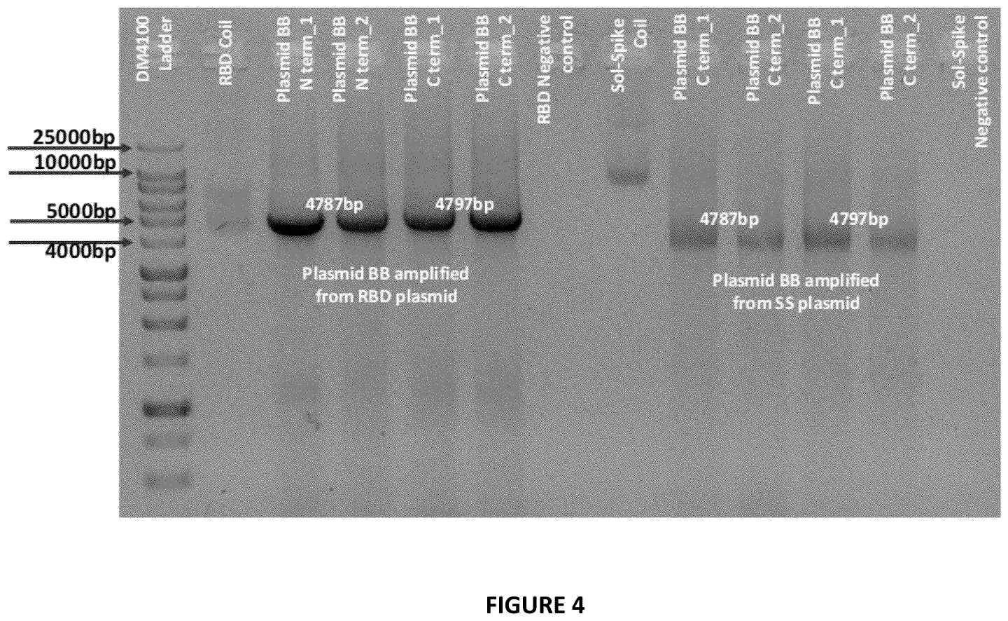

Reference is made to FIG. 1B, which is a simplified illustration comprising the steps of the herein disclosed method, in some embodiments.

In some embodiments, a first step 200 comprises providing a sample comprising labeled molecules and suspect of comprising a first molecule.

In some embodiments, a second step 220 comprises contacting the sample comprising labeled molecules from first step 200 with a second molecule, wherein the second molecule is labeled with a second labeling agent.

In some embodiments, a second step 240 comprises determining, under flow conditions, the temporal localization of the first labeling agent and of the second labeling agent.

According to some embodiments, there is provided a method for determining the presence of a first molecule in a sample, wherein the first molecule has specific binding affinity to a second molecule, comprising the steps of: (a) labeling the molecules of sample suspect of comprising the first molecule with a first labeling agent; (b) contacting the labeled molecules from step (a) with the second molecule, wherein the second molecule is labeled with a second labeling agent; and (c) determining, under flow conditions, the temporal localization of the first labeling agent and of the second labeling agent.

In some embodiments, the labeling comprises using single or multiple dyes that is suitable or configured to bind of the first molecule in a sample and the second molecule, for detection and diagnostic purposes.

In some embodiments, any one of the first molecule and second molecule is selected from: a small molecule, a nucleic acid (e.g., oligonucleotide, polynucleotide, etc.), a peptide (e.g., a polypeptide, a protein, etc.), a saccharide (e.g., monosaccharide, oligosaccharide, polysaccharide, etc.), a lipid, and any combination thereof.

In some embodiments, the first molecule is a biomarker indicative of any one of: cancer, brain injury or disease, inflammation, or an infectious disease.

As used herein, the term “biomarker” refers to any compound capable of being measured, thereby indicates or correlates to biological state or condition.

In some embodiments, the first molecule is a protein. In some embodiments, the second molecule is a protein. In some embodiments, the first molecule and the second molecule are proteins.

In some embodiments, the first molecule is a polynucleotide. In some embodiments, the second molecule is a polynucleotide. In some embodiments, the first molecule and the second molecule are polynucleotide.

In the first molecule being a polynucleotide is a host polynucleotide or a pathogen polynucleotide. In some embodiments, a host polynucleotide comprises an intracellular polynucleotide or a cell-free and/or circulating polynucleotide.

In some embodiments, a polynucleotide comprises DNA, RNA, or a hybrid thereof.

In some embodiments, the first molecule is a nucleic acid, and the second molecule is a nucleic acid capable of hybridizing thereto. In some embodiments, the first molecule being a nucleic acid is obtained or derived from a cell, a tissue, an organ, or a subject, and the second molecule being a nucleic acid is a synthetic nucleic acid, e.g., a probe, or vice versa.

In some embodiments, a nucleic acid comprises a cell-free nucleic acid. In one embodiment, a cell-free nucleic acid comprises cell-free DNA (cfDNA).

In some embodiments, the first molecule is a small molecule, and the second molecule is a peptide, a polypeptide, or a protein, or vice versa. In some embodiments the first molecule is an antagonist of the second molecule, or vice versa.

In some embodiments, there is provided a method for determining the presence of a first protein in a sample, wherein the first protein has specific binding affinity to a second protein, comprising the steps of: (a) labeling the proteins of a protein sample suspect of comprising the first protein with a first labeling agent; (b) contacting the labeled proteins from step (a) with the second protein, wherein the second protein is labeled with a second labeling agent; and (c) determining, under flow conditions, the temporal localization of the first labeling agent and of the second labeling agent.

According to some embodiments, there is provided a method for determining the presence of a particle in sample, comprising the steps of: (a) contacting a sample suspected of comprising a particle with a labeled compound having specific binding affinity to the particle; and (b) determining the intensity of a signal generated by the labeled compound.

In some embodiments, determining comprises generating a three-dimensional image based on a modified light to provide the depth of any one of the first molecule, e.g., a protein, labeled with the first labeling agent and the second molecule, e.g., a protein, labeled with the second labeling agent.

In some embodiments, the method further comprises determining the number of counts of the detected signal derived from the sample and being above the predetermined threshold, compared to the background, wherein an increase of at least 5%, at least 15%, at least 25%, at least 50%, at least 75%, at least 100%, at least 150%, at least 250%, at least 400%, at least 500%, at least 750%, or at least 1,000%, in the number of counts of the detected signal derived from the sample and being above the predetermined threshold, compared to the background, is indicative of the presence of the particle in the sample, or any value and range therebetween. Each possibility represents a separate embodiment of the invention.

In some embodiments, an increase comprises 5 to 150%, 25 to 500%, 10 to 450%, 50 to 750%, 100 to 1,000%, 200 to 1,500%, 225 to 950%, 320 to 1,250%, or 70 to 1,100% increase. Each possibility represents a separate embodiment of the invention.

In some embodiments, a detection of a signal above a predetermined threshold provided by the background is indicative of the presence of the particle in the sample, thereby determining the presence of the particle in the sample.

In some embodiments, the method comprises determining a signal provided by a background sample, thereby providing a predetermined threshold. In some embodiments, a background sample is devoid of a mixture of molecules suspected of comprising a first molecule having specific binding affinity to a second molecule, as described herein. In some embodiments, a background sample comprises at least one or some of the molecules in a mixture of molecules suspected of comprising a first molecule having specific binding affinity to a second molecule, as described herein, excluding the first molecule. In some embodiments, a predetermined threshold encompasses the signal provided by any sample according to the herein disclosed method, as long as the sample is devoid of the first molecule having specific binding affinity to a second molecule.

In some embodiments, the signal of an unknown sample determined according to the herein disclosed method is relative to the predetermined threshold. In some embodiments, the predetermined threshold has a signal normalized value of 1. In some embodiments, a sample devoid of the first molecule will provide a signal of 1 or less, when the predetermined threshold signal is normalized to a value of 1. In some embodiments, a sample comprising the first molecule will provide a signal greater than 1, when the predetermined threshold signal is normalized to a value of 1.

In some embodiments, colocalization of the first labeling agent and the second labeling agent in at least two tie points is indicative of the presence of the first molecule, e.g., a protein, having specific binding affinity to the second molecule, e.g., a protein, in the sample, thereby determining the presence of the first molecule, e.g., a protein, in the sample.

In some embodiments, cases wherein the first labeling agent and the second labeling agent do not colocalize are indicative of the absence of the first molecule, e.g., a protein, having specific binding affinity to the second molecule, e.g., a protein, in the sample, thereby determining the absence of the first molecule, e.g., a protein, in the sample.

In some embodiments, molecules are imaged under stable interactions of the first molecule and the second molecule. In some embodiments, molecules are imaged without stable interactions of the first molecule and the second molecule. In some embodiments, colocalization signals are determined under stable interactions. In some embodiments, under stable interactions only colocalization signals are above background. In some embodiments, without stable interactions, unbound molecules are visible as well as colocalization.

In some embodiments, the first molecule being a protein is an antibody.

In some embodiments, the first molecule being a protein is a cytokine. In some embodiments, a cytokine comprises a pro-inflammatory cytokine. In some embodiments, a cytokine comprises an anti-inflammatory cytokine.

In some embodiments, the second molecule being a protein is an antigen. In some embodiments, the antigen comprises or consists of a viral antigen. In some embodiments, the antigen is recognized, bound, or both, by the first protein.

In some embodiments, the second molecule being a protein is an antibody.

In some embodiments, the first molecule being a protein is an antigen of a second molecule being an antibody. In some embodiments, the first molecule being an is recognized, bound, or both, by the second molecule being a protein, e.g., an antibody.

As used herein, the term “antigen” refers to a molecule being “targeted” by an antibody. In some embodiments, the antigen comprises a molecule or molecular structure of a pathogen. In some embodiments, the antigen is present on the outer surface of a pathogen.

In some embodiments, the second molecule being a protein comprises a wild type form of the protein. In some embodiments, the second molecule being a protein comprises a mutated form of the protein. In some embodiments, the mutated form of the protein comprises one or more mutations. In some embodiments, the mutation is a synonymous or nonsynonymous mutation. In some embodiments, the mutation is a missense mutation. In some embodiments, the mutation comprises any mutation suitable for labeling the second protein.

In some embodiments, the second molecule being a protein comprises a chimeric form of the protein.

As used herein, the term “chimera” encompasses any conjugate comprising two or more moieties, wherein the two or more moieties are bound to one another either directly or indirectly, and wherein the moieties are either derived from distinct origins or are not naturally bound to one another. In some embodiments, the two or more moieties have: distinct functions, originate or derived from different genes, peptides, genomic regions, or species, distinct chemical classification (e.g., a peptide and a polynucleotide, as exemplified herein).

In some embodiments, the chimera comprises the second protein bound directly or indirectly to an agent, wherein the agent is selected from: a nucleotide, an oligonucleotide, a polynucleotide, an amino acid, a peptide, a peptide, a protein, a small molecule, a synthetic molecule, an organic molecule, an inorganic molecule, a polymer, a synthetic polymer, or any combination thereof.

As used herein, the term “directly” refers to cases wherein the second protein is bound to the agent in a covalent bond.

As used herein, the term “indirectly” refers to cases wherein each of the second protein and the agent are bound to a linker or a spacing element and not directly to one another. In some embodiments, the second protein is covalently bound to the linker. In some embodiments, the agent is either covalently or non-covalently bound to the linker.

As used herein, the term “covalent bond” refers to any bond which comprises or involves electron sharing. Non-limiting examples of a covalent bond include, but are not limited to: a peptide bond, a glycosidic bond, an ester bond, and a phosphor diester bond.

As used herein, the term “non-covalent bond” encompasses any bond or interaction between two or more moieties which do not comprise or do not involve electron sharing. Non-limiting examples of a non-covalent bond or interaction include, but are not limited to, electrostatic, π-effect, van der Waals force, hydrogen bonding, and hydrophobic effect.

The term “linker” refers to a molecule or macromolecule serving to connect different moieties of the chimera, that is the second protein and the agent.

In some embodiments, the method further comprises introducing a mutation to a polynucleotide sequence encoding the second protein.

In some embodiments, the method further comprises a step of conjugating, fusing, expressing, or any combination thereof, of a chimeric polypeptide, comprising the second protein.

In some embodiments, introducing a mutation comprises the addition of a modified and/or a non-canonical amino acid. In some embodiments, a modified and/or a non-canonical amino acid is conjugated to a dye. In some embodiments, a modified and/or a non-canonical amino acid is capable of binding or attaching to a dye, e.g., such as by click chemistry.

Methods for mutating polynucleotides and/or polypeptides, as well as labeling peptides, are common and would be apparent to one of ordinary skill in the art. Non-limiting example of such sequence modification and subsequent labeling is exemplified hereinbelow.

As used herein, the terms “protein”, “peptide”, and “polypeptide” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides.

As used herein, the term “oligonucleotide” refers to a short (e.g., no more than 100 bases), chemically synthesized single-stranded DNA or RNA molecule. In some embodiments, oligonucleotides are attached to the 5′ or 3′ end of a nucleic acid molecule, such as by means of ligation reaction.

The terms “polynucleotide”, “polynucleotide sequence”, “nucleic acid sequence”, and “nucleic acid molecule” are used interchangeably herein. These terms encompass nucleotide sequences and the like. A polynucleotide may be a polymer of RNA or DNA that is single- or double-stranded, that optionally contains synthetic, non-natural or altered nucleotide bases.

The term “small RNA” as used herein refers to short non-coding RNA molecules, including but not limited to microRNAs (miRNAs), small interfering RNAs (siRNAs), small nuclear RNAs (snRNAs), small nucleolar RNAs (snoRNAs), small temporal RNAs (stRNAs), antigen RNAs (agRNAs), piwi-interacting RNAs (piRNAs) and other short -regulatory nucleic acids.

The term “hybridization” or “hybridizes” as used herein refers to the formation of a duplex between nucleotide sequences which are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T (U), G and C of one sequence is then aligned with a T (U), A, C and G, respectively, of the other sequence. RNA sequences can also include complementary G=U or U=G base pairs. Thus, two sequences need not have perfect homology to be “complementary” under the invention.

In some embodiments, the first label comprises or is a bioluminescent label. In some embodiments, the second label comprises or is a bioluminescent label. In some embodiments, the first label and the second label comprise or are bioluminescent labels.

In some embodiments, the first molecule and the second molecule are labeled with the same labelling agent.

The term “bioluminescence” refers to the emission of light by biological molecules, such as proteins. Bioluminescence involves a molecular oxygen, an oxygenase, and a luciferase, which acts on a substrate, e.g., luciferin.

In some embodiments, a bioluminescent label comprises or is a fluorescent label.

As used herein, the terms “fluorescence” or “fluorescent agent” refers to any compound the emits light after it has absorbed light or other electromagnetic radiation.

As used herein, “flow conditions” encompasses “fluid communication” meaning fluidically interconnected and refers to the existence of a continuous coherent flow path from one of the components of the system to the other if there is, or can be established, liquid and/or gas flow through and between the ports, when desired, to impede fluid flow therebetween.

In some embodiments, the flow is a steady flow. In some embodiments, the flow is an unsteady flow. In some embodiments, the flow is a uniform flow. In some embodiments the flow is a non-uniform flow. In some embodiments, the flow is a steady and uniform flow. In some embodiments, the flow is a compressible flow. In some embodiments, the flow is an incompressible flow. In some embodiments, the flow is a one-dimensional flow. In some embodiments, the flow is a two-dimensional flow. In some embodiments, the flow is a three-dimensional flow. In some embodiments, the flow is a natural flow. In some embodiments, the flow is a forced flow. In some embodiments, the flow is a laminar flow. In some embodiments, the flow is a turbulent flow. In some embodiments, the flow is an internal flow. In some embodiments, the flow is an external flow. In some embodiments, the flow is a viscous flow. In some embodiments, the flow is a non-viscous flow.

In some embodiments, flow comprises diffusion.

In some embodiments, flow conditions comprise microfluidics.

As used herein, the term “microfluidics” encompasses any device which applies fluid flow to paths, e.g., channels, being smaller than 1 mm in at least one of their dimensions.

In some embodiments, the sample is derived from a subject.

In some embodiments, the sample is an environmental sample. In some embodiments, the sample is obtained, derived, collected, sampled, or any combination thereof, from an environment. In some embodiments, a sample derived or obtained from an environment is derived or obtained from: sewage, a water source, soil, or any combination thereof.

In some embodiments, the sample comprises any one of: bodily fluid, cell, tissue, biopsy, organ, and any combination thereof, derived or obtained from the subject.

As used herein, the term “bodily fluid” encompasses any fluid obtained from a living organism.

In one embodiment, bodily fluid comprises serum. In one embodiment, bodily fluid comprises plasma. Other non-limiting examples for bodily fluids include, but are not limited to, vitreous fluid, lymph fluid, synovial fluid, follicular fluid, seminal fluid, amniotic fluid, milk, whole blood, urine, cerebrospinal fluid, saliva, sputum, tears, perspiration, mucus, including tissue extracts such as homogenized tissue, and cellular extracts. In some embodiments, the sample comprises a biopsy. In some embodiments, the biopsy is obtained or derived from the gastrointestinal tract. In some embodiments, the sample comprises an epithelial cell derived from a subject. In some embodiments, an epithelial cell comprises a respiratory epithelial cell. In some embodiments, a respiratory epithelial cell is derived from the upper respiratory system. In some embodiments, a respiratory epithelial cell is a ciliated columnar epithelial cell. In some embodiments, a respiratory epithelial cell is a ciliated pseudostratified columnar epithelial cell. In some embodiments, a respiratory epithelial cell is selected from: a ciliated cell, a goblet cell, a club cell, or an airway basal cell.

Methods for obtaining a biological sample is well within the capabilities of those skilled in the art.

In some embodiments, the determining step is performed in vitro or ex vivo. In some embodiments, in vitro and/or ex vivo is in a test tube or in a plate.

In some embodiments, the sample comprises serum or any fraction thereof, of or derived from the subject.

In some embodiments, the subject is exposed or is suspected of being exposed to an infectious agent. In some embodiments, the infectious agent is selected from: a virus, a bacterium, a fungus, a unicellular parasite, a microparasite, or any combination thereof.

In some embodiments, the subject is exposed or is suspected of being exposed to a viral infection. In some embodiments, the subject is suspected of being infected with a virus. In some embodiments, the subject is exposed or is suspected of being exposed to a bacterial infection. In some embodiments, the subject is suspected of being infected with a bacteria. In some embodiments, the subject is exposed or is suspected of being exposed to a fungal infection. In some embodiments, the subject is suspected of being infected with a fungus. In some embodiments, the subject is exposed or is suspected of being exposed to a unicellular parasite infection. In some embodiments, the subject is suspected of being infected with a unicellular parasite. In some embodiments, the subject is exposed or is suspected of being exposed to a macroparasite infection. In some embodiments, the subject is suspected of being infected with a macroparasite.

In some embodiments, the subject is afflicted with inflammation.

As used herein, the term “inflammation” encompasses any response comprising immune cells and/or blood vessels and/or other molecular mediators, taken by the body to protect from pathogens, damaged cells, or any other harmful stimuli.

In some embodiments, the subject is afflicted with a disease. In some embodiments, the subject is afflicted with an injury.

In some embodiments, injury comprises trauma.

In some embodiments, the disease comprises cancer.

As used herein, the term “cancer” refers to a disease associated with cell proliferation, wherein the cell proliferation is abnormal, unregulated, dysregulated, or any combination thereof.

In some embodiments, disease, injury, or both, comprises brain disease, brain injury, or both.

In some embodiments, the method provides determination whether a subject is currently being infected with a virus, e.g., by determining the presence of a viral particle in a sample derived from the subject, e.g., a sample comprising epithelial cells of the subject, such as obtained by a swab.

In some embodiments, the method provides determination whether a subject was previously exposed to or infected with a virus, e.g., by determining the presence of an antiviral antibody in a sample derived from the subject, e.g., a sample comprising the serum or a fraction thereof. In some embodiments, an antiviral antibody is affecting or targeting a viral protein or a peptide. In some embodiments, the viral protein or peptide is Spike 1 protein or a fragment thereof.

In one embodiment, a virus is a SARS-Cov2 virus.

As used herein, the terms “subject” or “individual” or “animal” or “patient” or “mammal,” refers to any subject, particularly a mammalian subject, for whom therapy is desired, for example, a human.

As used herein, the term “specific binding affinity” refers to is binding with a dissociation constant (KD) ranging from 0.1 to 50 nM.

In some embodiments, increased binding affinity is binding with a dissociation constant (KD) of 0.1 nM at most, 0.5 nM at most, 1 nM at most, 5 nM at most, 7.5 nM at most, 10 nM at most, 15 nM at most, 20 nM at most, 25 nM at most, 30 nM at most, 35 nM at most, 40 nM at most, 45 nM at most, or 60 nM at most, or any value and range therebetween. Each possibility represents a separate embodiment of the invention.

In some embodiments, increased binding affinity is binding with a dissociation constant (KD) of 0.1 to 1 nM, 0.5-5 nM, 1-10 nM, 7-15 nM, 12-25 nM, 17-35 nM, 20-45 nM, 32-55 nM, 45-65 nM, or 40-70 nM. Each possibility represents a separate embodiment of the invention.

Methods for determining binding affinity and/or KD are common and would be apparent to one of ordinary skill in the art. A non-limiting example for a method of KD determination includes, but is not limited to, enzyme-linked immunosorbent assay (ELISA).

In some embodiments, the particle comprises a virus or a viral protein.

In some embodiments, the protein is a receptor or comprises a ligand binding domain.

In some embodiments, the labeled molecule comprises a ligand of the protein and a dye.

In some embodiments, the dye comprises a fluorescent dye.

According to some embodiments, the present invention utilizes microscopy so as to temporally determine the interaction and/colocalization under flow conditions of two compounds. In some embodiments, the microscopy is a 2-dimensional microscopy. In some embodiments, the microscopy is 3-dimensional microscopy.

In some embodiments, the herein disclosed method comprising temporal determination under flow conditions enable the tracking of at least one interaction/colocalization event over time, thereby provides increased sensitivity, accuracy, validity, or any combination thereof.

In some embodiments, the herein disclosed method comprising temporal determination under flow conditions enable the tracking of a plurality of interaction/colocalization events over time, thereby provides increased sensitivity, accuracy, validity, or any combination thereof.

In some embodiments, the herein disclosed method comprising temporal determination under flow conditions enable the tracking of a plurality of interaction/colocalization events, thereby provides increased sensitivity, accuracy, validity, or any combination thereof.

According to some embodiments, the present invention is directed to three-dimensional (3D) localization of individual objects over a customizable depth range in optical microscopy. In some embodiments, a conventional microscope is modified, and the shape of a point-spread-function (PSF) is used to encode the axial (depth) position of an observed object (e.g., a particle), and/or the color of the emitted light. The PSF is modified by Fourier plane processing using a phase mask, which is optimized for a depth-of-field range for the imaging scenario. An object, as used herein, includes an emitter, such as a particle, a molecule, a cell, a quantum dot, a nanoparticle, etc.

Single Particle Tracking (SPT) techniques are typically based on frame-by-frame localization of the particle. Namely, a series of time-sequential images (frames) are captured using a microscope, and each frame is analyzed to yield the current position of the particle. In some applications, the extracted positions are in two dimensions (2D), comprising lateral, or x,y coordinates, as well as color by dividing the field of view to two differentially illuminated regions. The noisy and pixelated 2D detector image of the particle is analyzed, e.g., by centroid or Gaussian fitting, to yield the estimated x, y coordinates of the particle. However, as many samples of interest are inherently three-dimensional (3D), the full physical behavior of the tracked object is revealed by analyzing its 3D trajectory. The 3D trajectory of a moving particle can be extracted in several ways. For example, a particle can be followed by using a feedback control loop based on moving a 3D piezo stage according to the reading of several detectors (e.g., photodiodes). While providing a very precise temporal and spatial trajectory, this method is inherently limited to tracking a single particle.

Alternatively, scanning methods, such as confocal microscopy, are implemented, in which an illumination beam or the focal point of the microscope (or both) are scanned over time in three dimensions to yield a 3D image of the object. Scanning methods are limited in their temporal resolution, since at a given time only a small region is being imaged. In order to simultaneously track several particles in 3D, a scan-free widefield approach can be used.

In some embodiments, 3D microscopic localization of point-like light objects is generated using wide-field microscopy. When a point-like (e.g., sub-wavelength) source of light is positioned at the focal plane of a microscope, the image that is detected on the imaging circuitry, such as a camera and/or a detector, is known as the PSF of the microscope. A conventional microscope's PSF (e.g., essentially a round spot) is used for imaging a two-dimensional (2D) ‘slice’ of a specimen, and for 2D (x,y) transverse localization of an object within that slice. That is, by fitting the shape of the spot with a 2D function such as a centroid, Gaussian, or Airy function, in some instances, the position of the object is detected with precision (a process termed super-localization). However, objects that are a small distance above or below the microscope's focal plane can appear blurry, and furthermore, their depth (or axial distance from the focal plane) is difficult to determine from their measured image. In some embodiments, 3D (x, y, and z) position information is obtained, even when an object is above or below the focal plane. Using a phase mask, an additional module is installed on a conventional microscope to solve the blur and depth issues. Instead of a point of light forming a single ‘spot’ on the camera, light passing through the phase mask forms a shape on the camera that looks different as a function of the object and distance from the focal plane (or amount of defocus).

In some embodiments, the method utilizes an optimization technique including PSFs with impressive depth ranges. Surprisingly, for a given optical system (e.g., with limitations defined by an objective lens), depth ranges are realized, for an application, far beyond previously known range limits of 2-3 μm. As a specific non-limiting example, using a phase mask optimized for a particular depth range, super-localization over a customizable depth range is performed up to 20 μm using a 1.4 numerical aperture (NA) objective lens. The depth range, for example, is a function of the NA objective lens and the light emitted by the object. In some embodiments, the PSF is used for 3D super-localization and tracking, as well as for 3D super-resolution imaging in biological samples, since this is an applicable depth range used for observing the 3D extent of a mammalian cell.

Certain PSFs, may be referred to as tetrapod PSFs, due to the shape they trace out in 3D space, as a function of the emitter position (the position of the object). In a number of embodiments, the modified shape characterizes the light as having two lobes with a lateral distance that changes along a line, having a first orientation, as a function an axial proximity of the object to the focal plane, and the line having a different orientation depending on whether the object is above or below a focal plane. In some embodiments, the different orientation of the line as compared to the first orientation, includes a lateral turn of the line from the first orientation to the different orientation, such as a 90 degree or 60 degree lateral turn. This shape has lines from the center of a tetrahedron to the vertices, or like a methane molecule. The PSF is composed of two lobes, where their lateral distance from one another and orientation are indicative of the z position of the object. Above the focal plane, the two lobes are oriented along a first line, and below the focal plane the two lobes are oriented along a second line that is differently orientated than the first line (e.g., perpendicular to the first line). For example, the modified shape is created, by decreasing the lateral distance (e.g., moving together) of the two lobes along the first line when the object is above the focal plane and is closer to the focal plane (e.g., moving closer), turning the two lobes laterally, such as 90 degrees, and increasing the lateral distance (e.g., moving apart) of the two lobes another along the second line when the object is below the focal plane and is further away from the focal plane (e.g., moving away).

Emitter (e.g., object) localization can be optimally performed using maximum likelihood estimation, based on a numerical or experimentally obtained imaging model. However, other localization methods can be used. While other methods for 3D imaging can be used, such methods use scanning (e.g. confocal), in which temporal resolution is compromised, or parallelizing the imaging system (multi-focal imaging), which complicates the implementation. In some embodiments, the method comprises observation of multiple single emitters in a field at high precision throughout depth ranges, such as discussed above.

In some embodiments, the method utilizes 3D super-localization microscopy techniques. Such techniques can include tracking single biomolecules with fluorescent labels inside a biological sample, and 3D analysis using other light emitting objects such as quantum-dots or the scattered light from gold beads or nano-rods. In some embodiments, the method comprises the use of a microfluidic device to characterize flow in 3D. In some embodiments, the method of the invention mitigates background noise in the measured image that is caused by fluorescent emitters that are outside the focal plane being optically excited, and therefore emit light (which contributes to background noise in the measured image). One method to mitigate background noise includes light-sheet microscopy (LSM). In LSM, only a narrow slice of the thick sample is illuminated at a given time, therefore only objects (e.g., emitters) within that slice are active (illuminating).

In some embodiments, an LSM (e.g., a relatively simple LSM) is used in combination with a tetrapod PSF. For example, with a tetrapod PSF, depth information is encoded in the PSF shapes, and the sample is illuminated in a descending angle relative to the field of view. The z-slice illuminated by the LSM is not parallel to the focal plane of the object, but rather, it is tilted by some angle. Due to the large depth range, PSFs in accordance with the present disclosure can accommodate an angle that is steep (tens of degrees). Therefore, imaging is performed all the way down to the substrate, and the light sheet is scanned. The tetrapod PSF, as used herein, is not a rotation of a shape of the passing light (e.g., relative to a center line) as a function of the axial position of the object (as with a spiral and/or helix PSF). Such embodiments can be advantageously implemented relative to previous LSM schemes. Such previous LSM schemes can be difficult to implement because imaging that is close to the bottom of the sample involves overlapping the illumination beam with the underlying glass substrate, which distorts the beam and prevents the formation of an undistorted light-sheet illumination profile. Therefore, LSM techniques (Bessel beam methods, for example) are cumbersome, costly, or use stringent manufacturing constraints. In one dual-objective design based on 45-degree excitation and collection objectives, the imaging is constrained to using low numerical aperture (NA) objective lenses, limiting the photon collection efficiency, and ultimately reducing precision.

According to some embodiments, the method comprises encoding an axial (e.g., depth) position of an observed particle by modifying a point-spread-function (PSF) using one or more parameterized phase masks. In some embodiments, each of such parameterized phase masks are optimized for a target depth-of-field range for an imaging scenario. In some embodiments, the optics pass light from an object toward the image plane and the phase mask. The phase mask is used to modify a shape of light, passed from the object. The shape modification includes a shape of light as a function of an axial proximity of the object, such as a tetrapod PSF. In various embodiments, the shape of light is characterized by having two lobes with a lateral distance that changes along a line, having a first orientation, as a function of an axial proximity of the object to a focal plane, and with the line having a different orientation depending on whether the object is above or below the focal plane.

In some embodiments, the shape modification includes a shape of light as a function of color, as previously described in Shechtman et al., 2016, (Letter to Naturephotonics) and in U.S. Pat. No. 10,341,640 B2.

The circuitry infers depth information about objects that are imaged. For example, the circuitry can be configured to infer depth of portions of the object based on the modified shape and a degree of blur, a tetrapod point-spread function (PSF), a 3D shape of the object on the image plane and a location of a portion of the object from which the light is emitted, and/or a Zernike polynomial (and any combination thereof). In some embodiments, the circuitry generates the 3D image based on a Zernike polynomial of at least a 3^{rd} order.

The phase mask, in some embodiments, is a deformable mirror used to tune the depth characteristic by deforming. For example, the phase mask tunes a depth characteristic to obtain light from the object at different respective depths. In some embodiments, the apparatus and/or method, as described above, includes a tuning circuit used to tune the depth characteristic.

Alternatively, instead of using a phase mask, a spatial light modulator (SLM) may be applicable.

In some embodiments, the method is used to track objects. For example, the method is used to localize an object, colocalize objects, e.g., 2 proteins (such as an antibody and an antigen thereof), track locations and/or movement of an object, track locations and/or movement of multiple objects simultaneously, and/or characterize flow in 3D in a microfluidic device (and any combination thereof).

In some embodiments, combining a tetrapod PSF with a tilted light-sheet microscope allows for depth measurements of individual fluorescing molecules over a depth range that reaches or exceeds 20 μm. This data is used to construct a 3D image of a large biological structure (e.g., whole mammalian cell) with resolution surpassing the diffraction limit by an order of magnitude. In the context of single-particle tracking microscopy, the phase mask allows for the 3D position of individual sub-diffraction limited objects to be monitored.

In some embodiments, phase mask design parameters may be adjusted to deliver optimal performance for a given depth range. Thereby, the phase mask in accordance with the present disclosure is not as limited in depth range as other depth estimation techniques. A phase mask can allow for a high numerical aperture (NA) implementation for light-sheet-microscopy.

In some embodiments, 3D position information is extracted from a single widefield 2D image, by modifying the microscope's point spread function (PSF), namely, the image which is detected when observing a point source. Examples of PSF alterations which are used for 3D tracking and imaging under biological conditions include astigmatism, the double-helix PSF, the corkscrew PSF, the bisected-pupil PSF, and an Airy-beam-based PSF, with applicable z-ranges of around 1-2 μm for astigmatism and the bisected pupil PSF, and around 3 μm for the double-helix, corkscrew, and Airy PSFs.

In some embodiments, generating (information optimal) PSFs for 3D imaging is based on numerically maximizing the information content of the PSF. Surprisingly, the resulting PSF exhibits superior 3D localization precision over other PSFs. Despite gradual improvements in PSF designs, other PSFs can be limited in terms of their applicable z-range. Currently, the z-range of other PSF designs is limited to around 3 μm, posing a major limitation for applications requiring ‘deep’ imaging. For example, the thickness of a mammalian cell can be larger than 6 μm and in the case of cells grown on cell feeder layers or in 3D cell cultures, which are becoming increasingly popular in the biological community, samples are much thicker than 3 μm.

In some embodiments, by utilizing the information maximization framework, a group or family of (tetrapod-type) PSFs are used for 3D localization over a depth range far larger than the applicable depth ranges of other designs, such as optimized for ranges of 2-20 μm. By setting the optimization parameters to correspond to the desired depth range, specific PSFs yield 3D localization optimized over the range. In some embodiments, a tetrapod PSF can be optimized for a 20 μm z-range, and as may be applicable to flow-profiling in a microfluidic channel. In some embodiments, such a PSF is optimized for a 6 μm z-range under biological conditions (e.g., tracking single quantum-dot labeled lipid molecules diffusing in live mammalian cell membranes).

Any concentration ranges, percentage range, or ratio range recited herein are to be understood to include concentrations, percentages, or ratios of any integer within that range and fractions thereof, such as one tenth and one hundredth of an integer, unless otherwise indicated.

Any number range recited herein relating to any physical feature, such as polymer subunits, size, or thickness, are to be understood to include any integer within the recited range, unless otherwise indicated.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference. Other general references are provided throughout this document.

### Materials and Methods

### Anti-Spike Antibody Detection by Single Molecule Co-Localization Fluorescence Microscopy Background

Detection of antibody in the serum using single molecule localization microscopy is done by labeling antibodies in the serum with one color, and labeling recombinant antigen with a different color, mix them and detect colocalization in the microscope. To test the applicability of this method the inventors evaluate the labeling of serum-like sample (containing antibodies) and antigen solutions using biochemical approaches (dot blot and western blot) and microscopy of static and flowing samples.

### Specific Aims

(1) To evaluate the binding stability between a fluorescent molecule and a protein; and (2) To determine the concentration ratios of labeling molecules to proteins.

### Procedure

### Antibody Labeling

SARS-CoV-2 (2019-nCoV) Spike S1 Antibody, Rabbit MAb (Sino Biological, 40150-R007), 100 μl of 1 mg/mL, divide into 20 μg aliquots-5 aliquots of 20 μl.

To one aliquot add 1 μl of ×20 BSA solution. This serves as a serum-like sample (Typical total protein concentration in serum is 60-80 mg/ml. (e.g., 343 mg/ml BSA in PBS, provided a final concentration of 17.2 mg/ml when adding 1 μl).

Mix N′ Stain CF568 labeling: follow manufacturer instructions with the following modification: (a) Add 2 μl reaction buffer ×10 to each 20 μl antibody sample; (b) Divide the Mix N′ Stain labeling solution (MX568S100-1KT, 50-100 μg) between the antibody solution and the serum like sample: ⅓ for the antibody solution, ⅔ for the serum-like sample; (c) Vortex and incubate for 30 min; and (d) Divide each sample to two aliquots and freeze.

### Antigen Labeling

Reconstitute lyophilized ‘THE His Tag Antibody (iFluor 647) mAb, Mouse, 100 μg, in 200 μl ddH₂O to give 0.5 mg/ml. divide into 7×28 μl aliquots.

Reconstitute the SARS-CoV-2 (2019-nCoV) Spike S1+S2 ECD-His Recombinant Protein (Sino Biological, 40589-VO8B1) in 400 μl ddH₂O and divide to 50 μl aliquots.

Incubate 28 μl antibody with 50 μl protein for 1 hr at room temperature or 4° C.

Divide into 4×19.5 μl

### Cross-Linking Anti His-Tag Antibody to Antigen

Glutaraldehyde—Treatment with crosslinkers should be conducted in buffers free from amines. Phosphate buffers at pH 7.5 to 8.0 and HEPES buffers are suitable whereas, Tris-HCl should be avoided. For glutaraldehyde treatment, reaction mixtures with 50 to 100 μg of interacting proteins in 20 mM HEPES buffer (pH 7.5) in a total volume of 100 μl are treated with 5 μl of 2.3% freshly prepared solution of glutaraldehyde for 2 to 5 minutes at, 37° C. The reaction is terminated by addition of 10 μl of 1 M Tris-HCl, pH 8.0.

### Antigen-Antibody Reaction

Mix 19.5 μl antigen with 5.5-16.2 μl labeled antibody or serum-like solution. Incubate for 1 hr.

### Microscopy

Sample preparation—Using super-resolution cleaned coverslip with square edge sticker, pipetting 2 μl sample in the center of the coverslip and covering with super-resolution cleaned coverslip.

Microscope—motorized inverted fluorescent microscope Ti2E, with ×100 silicon oil objective (CFI SR HP Plan Apochromat Lambda S 100XC 26).

Excitation—depending on the dye, with 561, 640, 488, 405 nm lasers, 10-50 mV. Using red, orange, and blue channels of a multichannel PSF engineering optical setup with tetrapod phase masks and appropriate filters (e.g. 650LP, on red channel).

Camera—Photomoterics prime 95B, 80 msec exposure time.

### Anti-Spike Antibody Detection by Spike-Ni-NTA Beads and Fluorescence Microscopy

### Background

Using Ni-NTA beads that bind his-tagged proteins to create virus-like particles (Sars-Cov2). The beads are added to a sample that contains labeled anti-spike antibodies (e.g., serum), the anti-spike antibodies bind the particle, and achieving strong signal over the background, thereby indicating their presence in the sample.

### Procedure

Samples included: 1. ECD (extracellular domain) spike protein labeled with anti-His antibody-iFluor555; 2. BSA-647; 3. ECD (extracellular domain) spike protein labeled with anti-His antibody-iFluor555 and anti-spike-647.

(1) Incubating 10 μl of 100 nm Ni-NTA beads with 10 μl of sample for 1.5 hr at RT. (2) Centrifuging at top speed for 1 min. Wash beads with 10 μl PBS. Centrifuging again, removing supernatant and resuspending in 10 μl PBS. (3) Diluting 1:100 with PBS. (4) Using super-resolution cleaned coverslip with square edge sticker, pipetting 2 μl sample in the center of the coverslip and covering with super-resolution cleaned coverslip.

### Microscopy

Microscope—motorized inverted fluorescent microscope Ti2E, with ×100 silicon oil objective (CFI SR HP Plan Apochromat Lambda S 100XC 26).

Excitation—depending on the dye, with 561, 640, 488, 405 nm lasers, 10-50 mV.

Using red, orange, and blue channels of a multichannel PSF engineering optical setup with tetrapod phase masks and appropriate filters (e.g. 650LP, on red channel).

Camera—Photomoterics prime 95B, 80 msec exposure time.

Cloning Spike Proteins with AviTag for Optimized Fluorescent Signal

### Background

The inventors use plasmids for two forms of recombinant spike proteins: (1) Receptor binding protein (RBD); and (2) Soluble spike protein, which comprises both S1 and S2 subunits and forms a trimer in solution. The inventors add AviTag to the proteins by cloning to allow fluorescent biotin labeled Qdot nanocrystals to be attached to the protein by BirA enzyme. This provides a bright signal for colocalization.

### Receptor Binding Domain (RBD) Cloning

Two gBlock's were ordered from IDT for RBD fused amino acid (AA) spacer and AviTag, on either N terminal or C-terminal. These fragments were ordered with restriction sites for XbaI upstream and for XhoI downstream.

(1) gBlock fragments was double digested with XbaI and XhoI (60 min in 37° C.) as well as pCAGGs seq with nCoV19 RBD plasmid for the backbone (BB); (2) gBlock product was cleaned using NucleoSpin; (3) Plasmid BB restriction product was verified using gel electrophoresis (1%) and purified from the gel using NucleoSpin; (4) Plasmid BB and gBlock products underwent ligation reaction (T4 ligase; 16° C. 18 hr incubation) and transformed into E. coli (DH5alpha); (5) Cloning was verified by sequencing; and (6) Verified clone with AviTag fused to the N′ terminus and clone with AviTag fused to the C′ terminus were grown to extract enough Plasmid DNA.

### Soluble Spike Cloning

(1) Two options of sequences encoding for Soluble Spike fused to two AA spacer and AviTag peptide was amplified using PCR, one option was fusion from the C′-terminus of Soluble Spike and the second option was fusion from the N′-terminus of RBD; (2) Soluble Spike PCR amplifications were performed using Q5 High-Fidelity DNA Polymerase, pCAGGs seq with nCoV19 soluble spike with CS deleted and PP mutation as a template, and appropriate primers which insert the AviTag and spacer: (a) For AviTag peptide addition in C′-terminus forward primer 5′-GCTAACCATGTTCATGCCTTCTTCTTTTTCCTACAGCTCCTGGGCAACGTGCTG GTTGTTGTGCTGTCTCATC-3′ (SEQ ID NO: 1) and reverse primer 5′-TTCATGCCATTCAATTTTCTGCGCTTCAAAAATATCGTTCAGGCCGCTGCCGTG ATGATGATGATGATGTCCC-3′ (SEQ ID NO: 2). (b) For AviTag peptide addition in N′-terminus forward primer 5′-ATGTCGGGCCTGAACGATATTTTTGAAGCGCAGAAAATTGAATGGCATGAAG GCAGCATGTTCGTGTTTCTGGTGCTG-3′ (SEQ ID NO: 3) and reverse primer 5′-CTTCATGATGTCCCCATAATTTTTGGCAGAGGGAAAAAGATCTGCTAGCTCGA GTCGCGACTTAAGATCGATGCGGCC-3′ (SEQ ID NO: 4); (3) Soluble Spike PCR product was verified using gel electrophoresis (1%); (4) Plasmid BB PCR amplifications were done using Takara PrimeSTAR® GXL DNA Polymerase, pCAGGs seq with nCoV19 RBD with His tag as a template, and appropriate primers which insert the AviTag and spacer: (a) For AviTag peptide addition in C′-terminus forward primer 5′-GGCAGCGGCCTGAACGATATTTTTGAAGCGCAGAAAATTGAATGGCATGAAT AATGAAATTCGAGCTCGCG-3′ (SEQ ID NO: 5) and reverse primer 5′-GATGAGACAGCACAACAACCAGCACGTTGCCCAGGAGCTGTAGGAAAAAGAA GAAGGCATGAACATGGTTAGC-3′ (SEQ ID NO: 6). (b) For AviTag peptide addition in N′-terminus forward primer 5′-CTTAAGTCGCGACTCGAGCTAGCAGATCTTTTTCCCTCTGCCAAAAATTATGG GGACATCATGAAGCCCC-3′ (SEQ ID NO: 7) and reverse primer 5′-GCTGCCTTCATGCCATTCAATTTTCTGCGCTTCAAAAATATCGTTCAGGCCCGA CATGGTGGCCTTTGCCAAAATG-3′ (SEQ ID NO: 8); (5) Plasmid BB PCR product was verified using gel electrophoresis (1%); (6) Plasmid BB and Soluble Spike PCR products were purified using NucleoSpin; (6) Appropriate plasmid BB and Sol_Spike fragments underwent Gibson assembly reaction to create closed plasmid containing Sol_Spike fused to 2 AA spacer and AviTag either on the C′- or N′-terminus, and transformed into E. coli (DH5alpha).

### Spike Protein Production in Mammalian Cells

### FreeStyle 293-F Cell Transfection-Branched Version of PEI

### Materials

Disposable Polycarbonate Erlenmeyer Flasks 125 ml/250 ml, pre-heated FreeStyle 293 expression medium, DNA (purified with Midipep kit), OptiMEM medium, and PEI reagent.

### Steps

(1) Seed cells at 0.7×10⁶ cells/ml into a final volume of 30 ml of pre-heated FreeStyle 293 expression medium in each 125 ml Erlenmeyer flask, 24 hours prior transfection; (2) Count the cells and determine the viability (for best results, make sure to have a single-cell suspension. Vortex may be required, e.g., vortex the cells vigorously for 10-45 seconds to break up cell clumps. The cells should reach a density of 1.0×10⁶ cells/ml (*If the cells are in a higher density—discard part of the cells and replace with a fresh FreeStyle 293 expression medium); (3) Pipette 37.5 μg of filter-sterilized DNA with OptiMEM and vortex vigorously for sec (Tube 1);

| | RBD-AviTag | RBD-AviTag |
|---|---|---|
| | N′-terminus | C′-terminus |
| Material | [1866.3 ng/μl] | [1569.6 ng/μl] |
| OptiMEM | 600 μl | 600 μl |
| DNA | 20 μl | 24 μl |

(4) Pipette 0.5 mg/ml filter-sterilized PEI (branched version) with OptiMEM to a final volume of 600 μl and vortex vigorously for 3 sec (Tube 2);

| | RBD-AviTag | RBD-AviTag |
|---|---|---|
| Material | N′-terminus | C′-terminus |
| OptiMEM | 480 μl | 480 μl |
| Branched PEI | 5 μl | 5 |
| (0.5 mg/ml) | | |

(5) Incubate both tubes for 5 min at RT; (6) Add tube 2 to tube 1 (and not tube 1 to tube 2); (7) Incubate the mix at RT for 15 min (do not wait longer than 20 min); (8) Add the DNA/PEI mix to the cells; and (9) Incubate the cells in an orbital shaker incubator for a further 48 hr at 37° C., 135 rpm, and 8% CO₂.

### His-Tagged Spike Protein Purification by Affinity Chromatography

(1) Collect the cell culture (or supernatant, in case of adherent cells) into 50 ml falcon; (2) Centrifuge at 4,000 g for 20 min at 4° C., discard the cell pellet (use aerosol-tight caps); (3) Filter the supernatant using 0.22 μm Stericap filter into 50 ml falcon; (4) Place the filtered supernatant on ice until use; (4) Wash Ni-NTA resin (600 μl per 20 ml culture) with 1.2 ml fresh PBS in a 2 ml Eppendorf tube; (5) Centrifuge at 2,000 g for 10 min, discard PBS; (6) Resuspend the resin with 1 ml filtered supernatant, transfer it into the falcon containing the rest of the supernatant and invert 3 times; (7) Incubate the resin with the supernatant for 2 hours on a roller shaker (Intelli mixer) at 10 rpm (C1 program) at room temperature (RT). Seal falcons with parafilm; (8) Load a clean polypropylene column with the supernatant-resin mixture; (9) Collect the flow-through in a 14 ml falcon; (10) Wash the 50 ml falcon with the flow through and re-load onto column (to make sure that all the resin is loaded, and to increase probability of protein binding); (11) Wash the column with ×10 column volume Wash buffer (3 ml Wash buffer); (12) Collect the wash solution in a 14 ml falcon; (13) Elute the protein with ×5 column volume Elution buffer (1.5 ml Elution buffer for 20 ml culture, 3000 for each fraction) into SafeSealed microcentrifuge tubes; and (14) Place the tubes on ice, or keep them at −80° C. (after freezing them in liquid nitrogen).

### Example 1

### Anti-Spike Antibody Detection by Single Molecule Co-Localization Fluorescence Microscopy

Recombinant ECD (Extra cellular domain) spike protein-iFluor555 was mixed with anti-spike antibody—CF640. The sample was diluted to ˜10⁻¹¹ M (of both antibody and spike protein) and mounted on a 0.17 mm coverslip. Microscopy setup included: 561 and 640 nm lasers; ×100 silicone oil objective; tetrapod phase mask (4 μm z range); and orange and red emission channels.

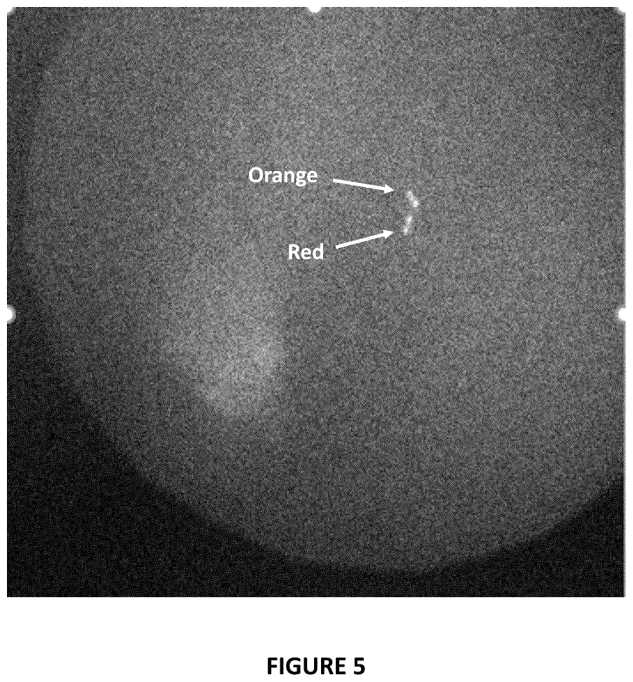

Single labeled spike protein was found to be attached to the anti-spike antibody (FIG. 5).

### Example 2

### Anti-Spike Antibody Detection by Single Molecule Co-Localization Fluorescence Microscopy in Flow

Total IgG antibodies in serum sample or PBS sample containing antibodies were fluorescently labeled with Zenon™ Human IgG Labeling Kit (either Alexa Fluor™ 488, 594 or 647). The sample was then mixed with ECD spike protein (labeled with different color than the Zenon™ Human IgG Labeling Kit used to label the antibodies), incubated, and mixed with glutaraldehyde to stabilize bound molecules and fluorescent signal. Using microfluidic system comprising of flow controller, low bind tubings, and a micro-channel, the sample was imaged in flow with a fluorescence microscope equipped with laser engine, 100×, NA=1.49 oil objective, phase masks in a color channel splitting system, and a sCMOS camera for counting colocalization events.

In some embodiments, at least some of the above steps and method may be performed by a computer-based system. The computer-based system may receive signals from the microscope, for example, a first signal at a first time point, indicative of a first temporal colocalization of a first labeling agent and of a second labeling agent and a second signal at a second time point, indicative of a second temporal colocalization of the first labeling agent and of the second labeling. The computer system may further determine the presence of the first molecule based on the first and second signals.

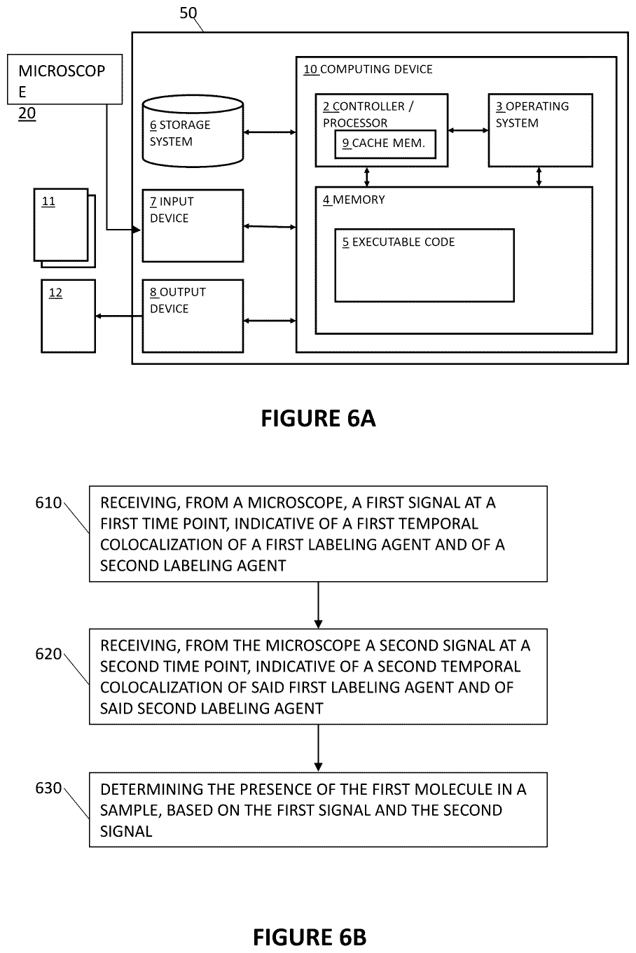

Reference is now made to FIG. 6A which is a block diagram of a computer-based system 50 according to some embodiments of the invention. System 50 may include a computing device 10. Computing device 10 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 10 may be included in, and one or more computing devices 10 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 10, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. For example, executable code 5 may be an application that may determine the presence of the first molecule in a sample as further described herein. Although, for the sake of clarity, a single item of executable code 5 is shown in FIG. 6A, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Any required data in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 10 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8. Input device 7 may be in communication with a microscope 20. Microscope 20 may be a microscope according to any embodiment of the invention disclosed herein above. A communication unit of microscope 20 may send signals to input device 7 to be processed by processor 2.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

Reference is now made to FIG. 6B which is a flowchart of a computer implemented method for determining the presence of a first molecule in a sample according to some embodiments of the invention. the method of FIG. 6B may be executed by processor 2 of system 50 or by any suitable processor. In step 610, processor 2 may receive from microscope 20 a first signal at a first time point, indicative of a first temporal colocalization of a first labeling agent and of a second labeling agent. In some embodiments, first labeling agent labels a first molecule and said second labeling agent labels a second molecule. For example, processor 2 may receive from microscope 20 a first SPT frame (e.g., the first signal) comprising the first temporal colocalization of a first labeling agent and of a second labeling agent.

In step 620, processor 2 may receive from microscope 20 a second signal at a second time point, indicative of a second temporal colocalization of a first labeling agent and of a second labeling agent. For example, processor 2 may receive from microscope 50 a second consecutive SPT frame (e.g., the second signal) comprising the second temporal colocalization of a first labeling agent and of a second labeling agent.

In step 630, processor 2 may determine the presence of the first molecule in a sample, based on the first signal and the second signal. For example, processor 2 mat determine the presence of an anti-spike antibody molecule according to examples 1 and 2.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for determining the presence of a first molecule in a sample, wherein said first molecule has specific binding affinity to a second molecule, the method comprising the steps of:
a. labeling molecules of a sample suspect of comprising said first molecule with a first labeling agent;
b. contacting said sample comprising said labeled molecules from step (a) with a second molecule labeled with a second labeling agent; and
c. determining, under flow conditions, the temporal localization of said first labeling agent and of said second labeling agent,
wherein colocalization of said first labeling agent and said second labeling agent in at least two time points is indicative of the presence of said first molecule having specific binding affinity to said second molecule in said sample, thereby determining the presence of the first molecule in the sample.

2. The method of claim 1, wherein said determining comprises generating a three-dimensional image based on a modified light path to provide the depth or color of any one of said first molecule labeled with said first labeling agent and said second molecule labeled with said second labeling agent.

3. The method of claim 1, wherein any one of said first molecule and said second molecule is selected from the group consisting of: a peptide, a nucleic acid, and a small molecule.

4. The method of claim 1, wherein said first molecule is a biomarker indicative of any one of: cancer, brain injury or disease, inflammation, and an infectious disease.

5. The method of claim 1, wherein said first molecule, said second molecule, or both, are proteins, optionally wherein any one of: (i) said first molecule being a protein is an antibody or a cytokine (ii) said second molecule being a protein is an antigen; (iii) said antigen comprises a viral antigen; and (iv) any combination of (i) to (iii).

6.-8. (canceled)

9. The method of claim 1, wherein said first molecule, said second molecule, or both, are polynucleotides.

10. The method of claim 9, wherein said first molecule being a polynucleotide comprises a host polynucleotide or a pathogen polynucleotide, and optionally wherein said polynucleotide comprises DNA, RNA, or a hybrid thereof.

11. (canceled)

12. The method of claim 1, wherein said first label, said second label, or both, are fluorescent labels.

13. The method of claim 1, wherein said flow conditions comprise microfluidics, diffusion, or both.

14. The method of claim 1, wherein said specific binding affinity is binding with a dissociation constant (K_{D}) ranging from 0.1 to 50 nM.

15. A method for determining the presence of a particle in sample, the method comprising the steps of:
a. contacting a sample suspected of comprising a particle with a labeled compound having specific binding affinity to said particle; and
b. determining the intensity of a signal generated by said labeled compound,
wherein a detection of a signal above a predetermined threshold provided by a background is indicative of the presence of said particle in said sample, thereby determining the presence of the particle in the sample.

16. The method of claim 15, further comprising determining the number of counts of said detected signal derived from said sample and being above said predetermined threshold, compared to said background, wherein an increase of at least 5% in the number of counts of said detected signal derived from said sample and being above said predetermined threshold, compared to said background, is indicative of the presence of said particle in said sample.

17. The method of claim 16, wherein said particle comprises a virus or a viral protein, optionally wherein said protein is a receptor or comprises a ligand binding domain, optionally wherein said labeled compound comprises a ligand of said protein and a dye, and optionally wherein said dye comprises a fluorescent dye.

18.-20. (canceled)

21. The method of claim 15, wherein said determining is under flow conditions, and optionally wherein said flow conditions comprise microfluidics, diffusion, or both.

22. The method of claim 1, wherein said sample is derived from a subject, and optionally wherein said sample derived from a subject comprises a cell, a tissue, and organ, a bodily fluid, or a fraction thereof, or any combination thereof, of said subject.

23. (canceled)

24. The method of claim 22, wherein said subject is exposed or is suspected of being exposed to an infectious agent.

25. The method of claim 24, wherein said infectious agent is selected from the group consisting of: a virus, a bacterium, a fungus, a unicellular parasite, and a microparasite.

26. The method of claim 22, wherein said subject is afflicted with a disease or an injury.

27. A system, comprising:
a processor; and
computer readable medium, having stored thereon instructions that when executed by the processor cause the processor to:
a. receive, from a microscope, a first signal at a first time point, indicative of a first temporal colocalization of a first labeling agent and of a second labeling agent, under flow, wherein said first labeling agent labels a first molecule and said second labeling agent labels a second molecule;
b. receive, from the microscope, a second signal at a second time point, indicative of a second temporal colocalization of said first labeling agent and of said second labeling agent, under flow; and
c. determine the presence of the first molecule in a sample, based on the first signal and the second signal.

28. (canceled)
